(19) United States
(12) Patent Application Publication
(10) Pub. No.: US 2014/0363879 A1
(43) Pub. Date: Dec. 11, 2014

(54) SYSTEM AND METHOD FOR SULFUR BIOADAPTATION IN A STABLE DISPERSION FORM FOR INDUSTRIAL BIOREACTOR FEEDING

(71) Applicants: Pilar Parada Valdecantos, Santiago (CL); Sebastian Galvez Avendano, Santiago (CL); Ricardo Badilla Ohlbaum, Santiago (CL); Francisco Laurie Santander, Santiago (CL)

(72) Inventors: Pilar Parada Valdecantos, Santiago (CL); Sebastian Galvez Avendano, Santiago (CL); Ricardo Badilla Ohlbaum, Santiago (CL); Francisco Laurie Santander, Santiago (CL)

(73) Assignee: BIOSIGMA S.A., Santiago (CL)

(21) Appl. No.: 14/122,541

(22) PCT Filed: May 24, 2012
(86) PCT No.: PCT/IB2012/052617

§ 371 Date: Aug. 27, 2014

(30) Foreign Application Priority Data
May 27, 2011 (CL) 1252-2011

(51) Int. Cl. C12N 1/20

(52) U.S. Cl. CPC C12N 1/20
USPC 435/252.1; 435/289.1

(57) ABSTRACT

A system for sulfur (S) bioadaptation in a stable dispersion form for feeding industrial bioreactors, which are composed of a tank with a liquid medium made up of a supernatant dilution or part of a sulfur oxidizing bacterial culture, a recirculating pump fed from the tank, and an eductor which is connected at its input to the recirculation pump, and connected at its output back to the tank, closing the recirculation circuit. There is a supply of S in fine powder form present in the eductor's feeding path. Where the supernatant or part of a sulfur oxidizing bacterial culture is diluted between 100 and 10 times in sulfur containing culture medium. The second aspect the invention discloses a method for sulfur bioadaptation in a stable dispersion form for feeding industrial bioreactors, which includes initiating recirculation through a recirculation pump, in a tank which contains a liquid medium made up of a supernatant dilution or part of a sulfur oxidizing bacterial culture; connecting a supply of fine sulfur powder to an eductor, which is connected to the recirculation pump at its input, and its output connected back into the reservoir. The incorporated sulfur is pulverized by the suction produced within the eductor.

### INVENTION FIELD

This invention discloses a system for sulfur bioadaptation in a stable dispersion form for industrial bioreactor feeding. In addition, it discloses a method for sulfur bioadaptation in a stable dispersion form for industrial bioreactor feeding.

### INVENTION BACKGROUND

Bioleaching is the most important process in biomining, and is defined as a method for solubilizing metals from complex matrices in acidic media, using direct or indirect microorganism action. Microorganisms which are useful for these processes can belong to either the Bacteria or Archaea domain, and can be classified according to their utility for specific processes. We can distinguish, for example, sulfur and iron oxidizing microorganisms, which are usually used for bioleaching.

For biomining operations, it is common practice to incorporate bioreactors to produce biomass. These bioreactors are either based on a liquid phase where bacteria proliferate (traditional reactors), or based on a solid packed bed with adherent bacteria through which a solution percolates (cell column reactor). Given the importance of the iron and sulfur oxidation processes of biomining bacteria, we can find bioreactors for iron and sulfur oxidizing microorganisms, the latter being the focus of this invention.

Among the sulfur oxidizing microorganisms, the most common and widely found in industrial processes involving sulfur or reduced sulfur compounds is Acidithiobacillus thiooxidans.

An essential step in cultivating these microorganisms is to feed the reactors with the bacteria's natural substrate, sulfur. The sulfur used is a fine powder, between 30 and 300 μm of size, and has various handling problems:
First, it is hydrophobic, so it cannot dissolve in the bioreactor's aqueous acid environment;
Second, when the fine powder is in contact with oxygen containing air, such as atmospheric air, it becomes flammable and explosive, which complicates the dosed feeding to reactors that needs to be a continuous feeding;
Finally, the large dimensions of industrial bioreactors implicate suspended load manipulation for the dosage of sulfur into reactors. If wind breezes or other similar phenomena occur, the feeding operations have to be suspended.

The first statement has important consequences in biomass generation. In any sulfur oxidizing bacterial culture, there is a latency period from the time point the inoculum and sulfur are added, until the exponential growth phase. This is largely due to the hydrophobicity of the sulfur, which means that it is not immediately bioavailable as a substrate for microorganisms.

There is a need for a sulfur feeding system which overcomes all these inconveniences.

The inventors have designed a system and a method for sulfur bioadaptation in a stable dispersion form or dense slurry for industrial bioreactor feeding. The obtained sulfur dispersion no longer has the flammable/explosive properties of fine sulfur powder. As a liquid, it can be incorporated into the tanks without the handling problems related with wind breezes or other similar phenomena. Surprisingly, this dispersion ensures immediate sulfur bioavailability to bacteria. This significantly reduces the lag phase and can shorten the biomass generation time in sulfur oxidizing bacteria bioreactors.

The invented system consists of incorporating fine sulfur powder through an eductor associated to a recirculation circuit, which is connected to an auxiliary tank. The liquid medium is a supernatant dilution or part of a sulfur oxidizing bacterial culture.

This system provides a homogenous sulfur liquid suspension that can be easily manipulated, thus avoiding the dangers of fine sulfur powder. The resulting sulfur dispersion can be used directly as the substrate for liquid phase reactors. In addition, if the sulfur dispersion is allowed to thicken and the resulting slurry is used to prepare the growth medium, it can be used as a substrate for packed bed reactors. Furthermore, the inventors have found that this dispersion or slurry allows for the immediate bioavailability of sulfur. If said dispersion or slurry is directly added as a substrate to a sulfur oxidizing bacteria biomass generating bioreactor, the lag phase is shortened, thus also shortening the biomass generation time.

### STATE OF THE ART

No document has been found in the state of the art which resolves the technical problem posed in the invention herein, which is to obtain sulfur in a stable dispersion form industrial bioreactor feeding.

Notwithstanding, sulfur powder problems powder have been recognized in the state of the art, such as its flammable and explosive nature. For example, U.S. Pat. No. 3,779,884 (1974) discloses a method for sulfur dispersion for agricultural purposes. Here, aqueous phase turbulence is used to atomize sulfur and generate fine dispersion in water. The system contains a recycling design where the sulfur is passed through pumps which exert dispersion. The invention herein differs from U.S. Pat. No. 3,779,884 because it employs a liquid medium composed of a supernatant dilution or part of a sulfur oxidizing bacterial culture instead of water. In addition, it uses an eductor system to generate the dispersion.

These differences produce a bioadaptable sulfur dispersion which is stable in time and decants as a fine powder that can be resuspended by mechanical stirring. On the other hand, the dispersion is not stable in water and as stirring ceases, the sulfur remains floating on the water surface, generating macroscopic sulfur flocs.

Subsequently, we found U.S. Pat. No. 4,372,872 (1983), which describes how to produce a fine sulfur suspension by injecting molten sulfur into an aqueous stream with vigorous stirring. However, the same document mentions how difficult it is to prepare a sulfur dispersion in water as it agglomerates. In addition, it indicates that external surfactants would be helpful but does not identify them, nor incorporates them into the process.

U.S. Application 2003/0185637A1 (2003) describes a method for transporting sulfur granules which contain sulfur powder, and the removal of said powder. The method consists using an eductor system to pump water at the sulfur granules to produce an aqueous solution which contains sulfur granules and powder. Then, the water containing sulfur powder and other impurities is separated from the sulfur granule transporting water. Finally, the water is removed from the sulfur granules, and the dry sulfur granules are stored.

The eductor system is well known in the state of the art, and is used to incorporate liquid, gaseous or powder substances to a liquid composition. For example, U.S. Pat. No. 4,695,378 (1987) describes a system for treating mine water, consisting of an eductor to aerate and add powdered neutralizing agent to the treated water. In a second example, U.S. Pat. No. 6,988,823 B2 (2006) describes a system for “wetting fine solids” and indicates that insoluble compounds can produce a homogeneous dispersion by the described technique. The system incorporates an eductor to mix the powder and the aqueous stream. This document does not make use of recycling; instead it describes the use of internal stirring rods to mix solution/dispersion.

In conclusion, although there are some solutions to the transport of sulfur powder as a dispersion, these have been demonstrated in water, which is less stable than the dispersion of bioadaptable sulfur obtained with the system and method of the invention herein.

The technical problem posed in the invention, to obtain bioadaptable sulfur in a stable dispersion for feeding industrial bioreactors, has not been addressed in prior art. The uses described for fine powder sulfur slurries or dispersions in the analyzed documents are for the agricultural sector, where sulfur is used as a fungicide and is never considered as feed for reactors, or as a microbial growth medium, as it is done in the invention herein.

### SUMMARIZED DESCRIPTION OF INVENTION

The invention refers to a system for sulfur (S) bioadaptation in a stable dispersion form for feeding industrial bioreactors, which are composed of:
a) a tank with a liquid medium made up of a supernatant dilution or part of a sulfur oxidizing bacterial culture,
b) a recirculating pump fed from the tank, and
c) an eductor which is connected at its input to the recirculation pump, and connected at its output back to the tank, closing the recirculation circuit. There is a supply of S in fine powder form present in the eductor's feeding path.

Where the supernatant or part of a sulfur oxidizing bacterial culture is diluted between 100 and 10 times in sulfur containing culture medium.

The second aspect the invention discloses a method for sulfur bioadaptation in a stable dispersion form for feeding industrial bioreactors, which includes:
a) initiating recirculation through a recirculation pump, in a tank which contains a liquid medium made up of a supernatant dilution or part of a sulfur oxidizing bacterial culture;
b) connecting a supply of fine sulfur powder to an eductor, which is connected to the recirculation pump at its input, and its output connected back into the reservoir. The incorporated sulfur is pulverized by the suction produced within the eductor.

### BRIEF DESCRIPTION OF FIGURES

FIG. 1 shows a process diagram of the preferred configuration of the invention, and consists of: a tank (1) with liquid medium containing a supernatant dilution or part of a sulfur oxidizing bacterial culture; a recirculation pump (2) fed from the tank (1), and an eductor (3) connected at its input to the recirculation pump (2), and connected at its output back to the tank (1), closing the recirculation circuit, and where the eductor (3) contains, in its feeding path, a supply of fine sulfur powder.

FIG. 2 shows an process diagram of the second preferred configuration of the invention. In addition to the system described in FIG. 1, this configuration contains: at the output of the recirculation pump (2), a means for directing flow (4) that allows to alternatively direct the flow directly to the tank (1), without passing through the eductor.

FIG. 3 shows the preferred configuration for sulfur bioadaptation in industrial processes. This configuration considers the process diagrams described in FIGS. 1 and 2. However, it includes an industrial bioreactor that acts as a source of supernatant or culture dilution to obtain bioadaptable sulfur, which in turn, is intended for feeding the industrial bioreactor.

FIG. 4 shows sulfur dispersion aliquots in 1× KMD culture medium in three conditions: FIG. 4a) sulfur bioadaptation of the invention, the sulfur decants completely, FIG. 4b) control with mechanical dispersion, the sulfur does not form large clumps, a small portion is decanted and a large percentage remains at the surface in flocs formed by the aggregation of the suspended solid. FIG. 4c) control without mechanical dispersion, the sulfur remains on the surface in the form of large clumps.

FIG. 5 shows the evolution of the pH and biomass in 3 sulfur oxidizing bacteria bioreactors inoculated with 106 cells/ml of Licanantay strain DSM 17318, where the control (▪) was fed with normal fine sulfur powder (non-bioadaptable), (▴) represents bioadaptable sulfur sterilized by autoclaving, and () represents sterilized bioadaptable sulfur. In FIG. 5.A, pH over time is shown, suggesting that bioadaptable sulfur lowers pH faster than the control. FIG. 5.B shows biomass over time, showing that bioadaptable sulfurs increase the biomass faster than the control, decreasing the lag phase from 75 h to 24 h.

FIG. 6 shows the percentage of conversion of sulfur to sulfuric acid in sulfur agglomerated on quartz columns with two different sulfur concentrations: 2.35 G_S/Kg_Quartz and 4.65 G_S/Kg_Quartz. Where the control (▴) was agglomerated with normal fine sulfur powder (non-bioadaptable), and the columns of the invention were agglomerated with bioadaptable sulfur (♦). FIG. 6A shows the percent conversion of sulfur to sulfuric acid for columns with lower sulfur concentrations 2.35 G_S/Kg_Quartz, while FIG. 6.B shows the same results for the column with higher sulfur concentrations, 4.65 G_S/Kg_Quartz. In both cases, the conversion of sulfur to sulfuric acid is faster and more efficient when using bioadaptable sulfur.

FIG. 7 shows bacterial growth in flasks with non-bioadaptable fine sulfur powder, FIG. 7A, and bioadaptable sulfur, FIG. 7B in the presence of different aluminum sulfate concentrations: 0 g/L (), 15 g/L (▴), 18 g/L (⋄) and 20 g/L (□). The toxic effect of the aluminum sulfate is diminished when using bioadaptable sulfur as a substrate.

### DETAILED DESCRIPTION OF INVENTION

As indicated in the invention background, cultivating sulfur oxidizing microorganisms requires feeding sulfur to the bioreactor. The sulfur is a fine powder, between 30 and 300 μm, and has a number of handling problems, such as its hydrophobicity and its flammable and explosive properties.

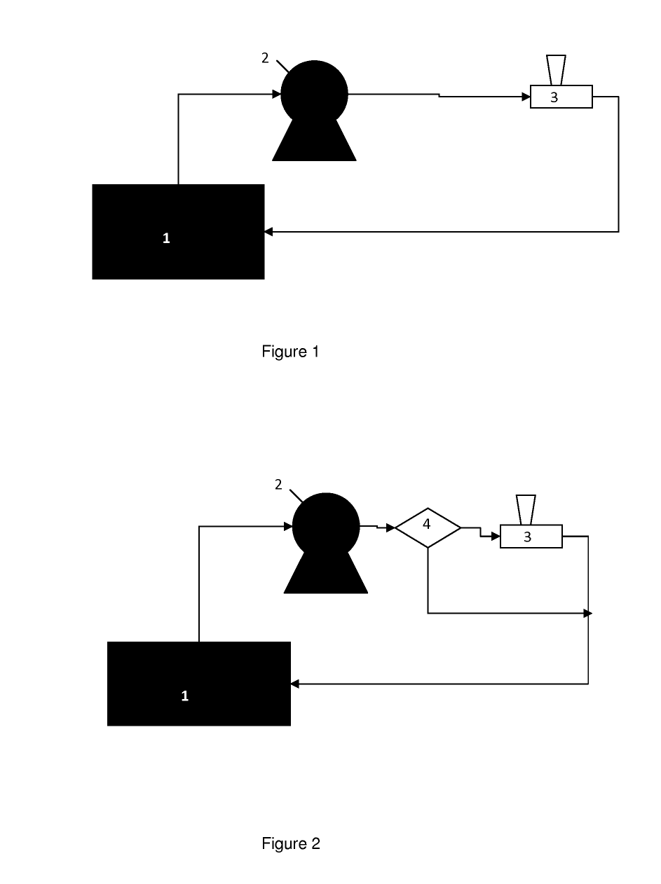

The inventors have designed a system and a method for sulfur bioadaptation in a stable dispersion form for industrial bioreactor feeding.
a) The invention refers to a system for sulfur bioadaptation in a stable dispersion form for industrial bioreactor feeding, and consists of: a tank (1) with a liquid medium containing a supernatant dilution or part of a sulfur oxidizing bacterial culture,
b) a recirculating pump (2) fed from the tank (1), and
c) an eductor (3) connected at its input to the recirculation pump (2) and connected at its output back to the tank (1), closing the recirculation circuit, and where the eductor's (3) feeding path contains a supply of fine sulfur powder.

Where the supernatant or part of a sulfur oxidizing bacterial culture is diluted between 100 and 10 times in the culture medium used to disperse the sulfur. This is represented in FIG. 1.

Furthermore, an alternative configuration of the invention consists of: placing at the recirculation pump's (2) output, a means for directing flow (4) which alternatively directs the flow directly to the tank (1), without going through the eductor. This preferred configuration is shown in FIG. 2.

In a second aspect, the invention reveals a method for sulfur bioadaptation in a stable dispersion form for feeding industrial bioreactors which consists of:
a) initiating recirculation within the tank (1), through a recirculation pump (2) which circulated liquid medium containing a supernatant dilution or a sulfur oxidizing bacterial culture, and
b) connecting a supply of fine sulfur powder to an eductor (3) which is connected to the recirculation pump (2) at its input, and connecting its output back into the tank (1).

Furthermore, for the preferred configuration of the invention herein, the method may consist of these additional steps:
c) connecting a means for directing flow (4) at the output of the recirculation pump (2) which allows to selectively direct the flow, and
d) selectively switching the flow, by the means for directing flow (4), to operate the eductor (3) through the recirculation flow or to direct the flow directly from the recirculation pump (2) to the tank (1).

Although the eductor's main function is to feed the system with sulfur powder, once all the powder has been introduced to the system, the eductor starts to introduce small air bubbles to the flow. This is due to the difference in pressure between the flow and the atmosphere. These bubbles generate turbulence in both the eductor discharge, and also within the tank, allowing for sulfur dispersion within the tank.

In the event that the valve directs the flow directly to the tank without passing through the eductor, and that all the sulfur powder has been introduced and has entered in forced contact with the supernatant, the flow characteristics due to the recirculation allow to obtain or maintain a stable sulfur dispersion due to the presence of “biosurfactant” compounds in the supernatant or in the sulfur oxidizing bacterial culture. Since an eductor ‘bypass’ exists, the pump avoids the hydraulic resistance of the eductor, which avoids the formation of sulfur flocs formed by the aggregation of fine sulfur in suspension.

Furthermore, the means for directing flow (4) may be a 3-way valve or a set of two valves each controlling flow entry to the corresponding circuit directions.

These two aspects of the invention, the system and method, very related in between them, allow a stable sulfur dispersion known as bioadaptable sulfur, which no longer has the characteristics of inflammable/explosive fine sulfur powder. The fact that it is a liquid means allows the incorporation of it into the tank without taking into account wind breeze problems. It can also be used to prepare a thick sulfur slurry for building packed beds of bacterial growth. Surprisingly, this sulfur dispersion or slurry ensures immediate bioavailability to microorganisms, and markedly decreases the lag phase. In addition, it can also shorten biomass generation in sulfur oxidizing bacteria bioreactors.

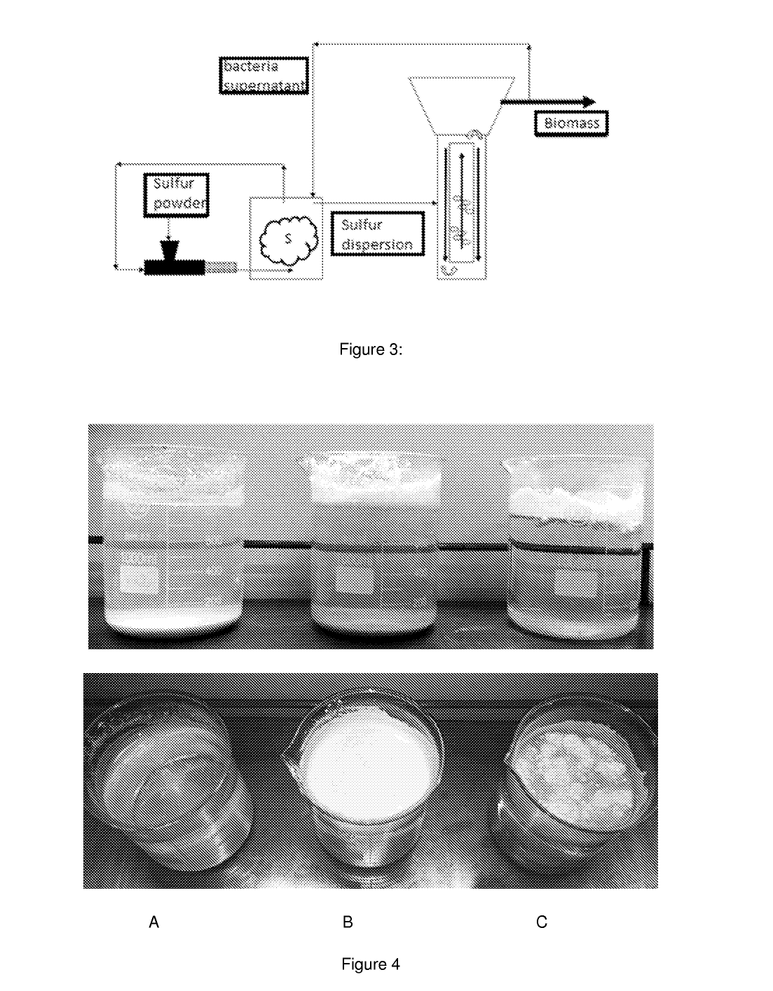

As seen in the state of the art analysis, reported sulfur dispersions included the use of water as a liquid carrier. Although the use of a surfactant was recognized to improve sulfur dispersion, this involved the addition of external agents to the culture that in some cases could prove harmful to microorganisms (Bouffard Sylvie. C., Tshilombo, Paul and West-Sells, Paul G., Mineral Engineering, 2009, Vol 92, 100-103). In an industrial context, the addition of external agents increases costs, making its widespread use difficult. Moreover, if the culture has to feed a mining operation, the addition of external agents is complicated by the fact that they could interfere with subsequent mining processes such as solvent extraction or electroextraction of the metal cathodes. The inventors solved this technical problem by using liquid medium or a supernatant dilution of a sulfur oxidizing bacterial culture, both of which are fully compatible with industrial processes. Where the supernatant or part of a sulfur oxidizing bacterial culture is diluted between 100 and 10 times in the culture medium used to disperse the sulfur. In this case, there is no addition of any external agent to the process. The preferred configuration at the industrial level is shown in FIG. 3.

It is known that supernatants, which correspond to the liquid medium in which bacteria grow once these are decanted, are highly complex solutions because they contain a range of biomolecules produced by bacterial metabolism. It is known that the supernatant of sulfur oxidizing bacterial cultures contain compounds with biosurfactant properties. Sulfur oxidizing bacteria, such as Acidithiobacillus thiooxidans, secrete phospholipids, where phospholipids in general, and phosphatidylethanolamine, phosphatidyl glycerol, phosphatidyl inositol in particular, have been cited in literature as agents produced by sulfur oxidizing bacteria which promote sulfur dispersion in water.

The inventors considered prior knowledge in order to develop the invention herein, as they recognized the real potential of sulfur oxidizing bacterial cultures for bioadapting sulfur. They coupled the dispersing advantages of biosurfactant compounds present in the supernatant of sulfur oxidizing bacteria, with a mechanical dispersion which accelerates the coating of sulfur with the surfactant compounds. This takes advantage of the synergy of both mechanisms to solve the inherent problems of sulfur's hydrophobicity.

As indicated, the elements needed to produce sulfur bioadaptation are found in the supernatant of the sulfur oxidizing bacterial culture. Notwithstanding, it is not necessary to have a pure supernatant, free of bacteria. The inventors have found that the effect of sulfur bioadaptation is achieved by using the supernatant, or by using the whole culture, which contains both the supernatant and the bacteria growing in it.

The characteristic composition of the supernatant of sulfur oxidizing bacterial culture, measured by the authors, contains:
Bacteria and growth products of the microorganisms, as in sulfur oxidizing bacteria such as Licanantay at concentrations of about 5×10⁹ cells/mL in a medium containing 10 g/L of sulfur.
Exopolysaccharides (EPS) used by bacteria for biofilm formation on sulfur, as for example a total of 20 mg/liter of EPS removable from a Licanantay culture grown in medium 5 g/L of sulfur. The EPS of Licanantay grown in sulfur is composed mainly of glucose, galactose, glucuronic acid, galacturonic acid, and arabinose.
Secreted proteins or the sulfur/bacteria interface, as for example 0.5 to 2 mg/L of proteins, mainly Licanantasa, as described in the patent application PCT WO2011024096, BioSigma S.A.
Metabolites resulting from cell metabolism: certain metabolites can be intentionally secreted, while others may be found in the supernatant due to cell lysis. For example, glutamic acid, aspartic acid, spermidine, proline, valine, and phenylalanine are the most abundant amino acids according to measurements taken by capillary electrophoresis coupled to mass spectrometry (CEMS) in supernatants.
Extracellular lipids: about 60% of extracellular lipids in a Acidithiobacillus thiooxidans culture, such as Licantantay bacteria, are neutral lipids, while the remaining 40% are phospholipids. Phosphatidylethanolamine, phosphatidylglycerol, and phosphatidylinositol, were detected in supernatants and their surfactant action on sulfur was tested (Beebe, James L. and Umbreit, W W, Journal of Bacteriology, 1971, Vol 108 (1), 612-614).

Some of the components present in the supernatant of sulfur oxidizing bacterial cultures have surfactant properties and are recognized as extracellular lipids. These are likely to be responsible for the sulfur bioadaptation observed in the system and method described for the invention herein. An advantage of this method and system is the use of the supernatant or the complete medium, since these facilitate the process operation.

### EXAMPLE 1

Sulfur Bioadaptation.

The sulfur bioadaptation system of the invention was set up according to the preferred configuration described in FIG. 2: a 50 liter tank (1) connected to a 2.5 Hp recirculation pump (2) fed from the tank (1), where a ¾″ (1.91 cm) eductor (3) is connected at its input to the recirculation pump (2) and connected at its output back to the tank (1), closing the recirculation circuit, and at the recirculation pump output (2), a means for directing flow was installed (4), in this case a 3-way valve, which allows to alternatively direct the flow directly to the tank (1), through a simple pipe.

This configuration maintains the tank in agitation, without using the eductor.

Once the system was installed, 3 experiments were performed:
1.1 Condition of the invention, bioadaptable sulfur. 38.8 L of 1× KMD culture medium were added, as described in Table 1, to the tank, with 1.2 L of Licanantay sulfur oxidizing bacterial culture (3% vol/vol in the KMD culture medium described in Table 1). The medium recirculation began through the eductor, which added in doses a total of 800 g of fine sulfur powder (concentration 20 g/L). Once the sulfur was added, the mechanical stirring was maintained in the tank. The recirculation went through the path that does not contain the eductor.

TABLE 1

| | | |
|---|---|---|
| (NH₄)₂SO₄ | 0.99 | g/L |
| NaH₂PO₄•H₂O | 0.128, | g/L |
| KH₂PO₄ | 0.0525 | g/L |
| MgSO₄•7H₂O | 0.1 | g/L |
| CaCl₂ | 0.021 | g/L |
| Agua | 1 | L |

KMD 1X Culture Medium Composition

After 15 minutes of having added sulfur, with continued stirring, an aliquot of 200 mL was removed in a beaker, in order to assess the condition visually. The results are shown in FIG. 3a) where one can observe that the sulfur is completely decanted. This decanted powder forms a stable dispersion in water when subjected to stirring.
1.2 Control with mechanical dispersion. 40 L of KMD 1× culture medium was added to the tank, as described in Table 1, with the initiation of the recirculation of the medium through the eductor, adding in doses a total of 800 g of fine sulfur powder (concentration 20 g/L). Once the sulfur was added, the mechanical stirring was maintained in the tank through the path that does not contain the eductor. After 15 minutes of having added the sulfur, with continued stirring, a 200 mL aliquot was removed in a beaker and the condition was assessed visually. The results are shown in FIG. 3b) which shows that the sulfur does not form large clumps, a small portion decants and a large percentage remains on the surface in a sulfur “foam” or “flocs” formed by the aggregation of the sulfur in suspension.
1.3 Control without mechanical dispersion. 200 mL of 1× KMD culture medium, as described in Table 1, and 4 g of fine sulfur powder (concentration 20 g/L) were added directly to a beaker, without agitation. After 15 minutes of having added the sulfur, the condition was assessed visually. The results are shown in FIG. 3c) where one can observe that the sulfur, because of its hydrophobic nature, remains on the surface where it forms large clumps.

The invention method to produce bioadaptable sulfur, situation 1.1, markedly improved the fine sulfur powder dispersion conditions in aqueous medium.

### EXAMPLE 2

The Use of Bioadaptable Sulfur as a Substrate in Sulfur Oxidizing Bacteria Bioreactors.

Three equal bubble column bioreactors were set up for the growth of sulfur oxidizing bacteria. The reactors have an effective volume of 1.7 L, which was supplemented with KMD 1× culture medium, whose components are shown in Table 1. Aeration pumps aerate the reactor with a flow of 3.5 L/min. The initial pH is 1.6. Each bioreactor is inoculated with 10⁶ cells/ml of Licanantay strain DSM 17318, Acidithiobacillus thiooxidans strain, BioSigma S.A. property, protected in the patent application CL 2101-2005 and in U.S. Pat. No. 7,700,343. The temperature in the reactors was maintained at 30° C.

10 g/L of fine sulfur powder was added to these bioreactors for the following conditions:
2.1 Control. Sulfur powder was added directly to the reactor as a substrate, at time 0.
2.2 Bioadaptable sulfur sterilized by autoclaving. In order to ensure that the observed effect is due to the dispersion bioadaptable sulfur achieved by the method of the invention, and not to a possible increase of the inoculum due to the culture medium used in the preparation bioadaptable sulfur, the sulfur is prepared according to the method of the invention and subsequently subjected to sterilization. Said bioadaptable sulfur sterilized by autoclaving is added to the second bioreactor as a substrate, at time 0.
2.3 Bioadaptable sulfur sterilized by sonication. As a second control, in order to ensure that the effect observed is due to the dispersion of bioadaptable sulfur obtained by the method of the invention, and not to a possible increase in the inoculum because of the culture medium used in the preparation of the bioadaptable sulfur, the sulfur is prepared according to the method of the invention and subsequently subjected to sterilization. Said bioadaptable sulfur sterilized by sonication is added to the third bioreactor as a substrate, at time 0.

These reactors were evaluated for biomass concentration and for medium pH. Sulfur oxidizing bacteria, such as Licanantay, metabolize sulfur to produce sulfuric acid and biomass. Thus, a greater production of sulfuric acid is indicative of an increased metabolism of sulfur by the bacteria. Sulfuric acid concentration can be easily estimated by measuring the pH of the culture medium, which decreases as the concentration of the acid increases. Moreover, the aim of these bioreactors is to increase biomass production, which was assessed as the concentration of biomass over time.

The results are shown in FIG. 4, where FIG. 4A shows the evolution of pH in the three bioreactors: bioreactors 2.2 and 2.3 have a faster decrease in pH between 50 and 200 hours of reaction. All three bioreactors reached the same pH.

FIG. 4B shows the biomass concentration. It can be seen that biomass grows rapidly in bioreactors with bioadaptable sulfur. At 24 hrs, there are differences of an order of magnitude compared to the control, which is maintained in the range of 10⁶ until approximately 80 hrs. At 80 hrs, the biomass in the reactors fed with bioadaptable sulfur of the invention is in the order of 10⁸-10⁹ cells/mL. Surprisingly, the lag phase of the culture when using bioadaptable sulfur lasts less than 24 hours. In the control with non-bioadaptable sulfur, however, the lag phase lasts 75 hours.

### EXAMPLE 3

Use of Bioadaptable Sulfur as a Substrate in Sulfuric Acid Generating Columns.

Four columns of agglomerated sulfur on quartz were set up with two different sulfur concentrations: 2.35 G_S/Kg_Quartz and 4.65 G_S/Kg_Quartz. Bioadaptable sulfur slurry was used to agglomerate 2 columns (one for each concentration), while non-bioadaptable fine sulfur powder was used for the two others, such that each sulfur concentration has two representative columns, a control with fine powder sulfur and one with bioadaptable sulfur. Each agglomerated sulfur on quartz received an aliquot of the same inoculum of 0.5 L_Inoculum/Kg_S, so that the ratio of sulfur/bacteria is constant for each reactor at time 0. The inoculum was Licanantay strain DSM 17318, Acidithiobacillus thiooxidans strain BioSigma S.A. property, protected in the patent application CL 2101-2005 and in U.S. Pat. No. 7,700,343. The cellular concentration of the inoculum was estimated by optical counting at 5 10⁹ cells/mL.

The bioadaptable sulfur of the invention is easily homogenized on a solid bed such as quartz, through hand agglomeration at a laboratory scale, or by homogenization in agglomeration drum at an industrial scale.

A solution of KMD 1×, at pH 1.6, was allowed to percolate in a temperature controlled chamber at 30° C., which resulted in suitable conditions for sulfur oxidizing bacterial growth, such as Licanantay. The columns have an approximate packed bed height of 10 cm and use 500 g of quartz each. The columns are aerated by diffusion of atmospheric gas, without forcing aeration. The percolation of the solution is controlled by peristaltic pumps adjusted to deliver a flow of 6 mL/hr to each column. Accumulated effluent was collected daily from each column, and the sulfuric acid concentration of the effluent was measured. As the beginning concentration of the sulfuric acid in the feed solution (influent) is known, the difference in the sulfuric acid concentration of the influent and the effluent represent the acid product of the sulfur metabolism produced by the present biomass.

The columns were then evaluated, assessing their production of sulfuric acid from sulfur. Given that sulfur oxidizing bacteria, such as Lincanantay, metabolize sulfur to produce sulfuric acid, an increased production of sulfuric acid means that there was increased sulfur metabolism by the bacteria. It is known that the metabolism of a unit of mass of sulfur produces about 3 units of mass of sulfuric acid. Said sulfur conversion to sulfuric acid is expressed in terms of conversion efficiency, as a percentage of sulfur that was converted into sulfuric acid, according to solution balances.

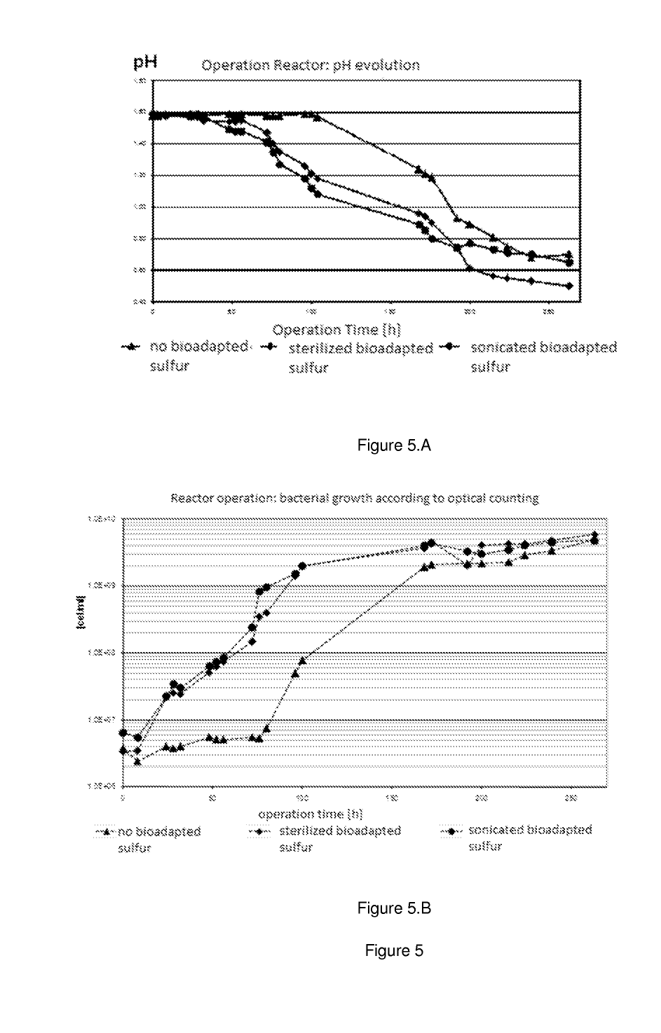

The results are shown in FIG. 5, where FIG. 5.A shows the percent conversion of sulfur to sulfuric acid for the columns with a lower sulfur concentration, 2.35 G_S/Kg_Quartz, while FIG. 5.B shows the same results for the column with the higher sulfur concentration, 4.65 G_S/Kg_Quartz. One can see that for the two columns producing sulfuric acid, the bioadaptable sulfur (♦) allowed 1) the early onset of the sulfuric acid in the effluent, 2) to increase production rate of sulfuric acid (estimated from the slope of the graph) and 3) allowed a better final sulfur conversion with respect to the non-bioadaptable fine sulfur powder (▴). The fact that the conversion does not reach 100% is mainly due to losses due to a drag of the sulfur in the effluents, and in the case of non bioadaptable sulfur, because of the formation macroscopic agglomerated sulfur.

### EXAMPLE 4

Use of Bioadaptable Sulfur as a Substrate in Flask Cultures of Sulfur Oxidizing Bacteria, with the Presence of a Toxic Agent that Inhibits Growth

Eight flasks with sulfur oxidizing bacteria in KMD 1× medium at 30° C. were prepared. Aluminum sulfate was added such that there were 4 aluminum concentrations: 0 g/L (), 15 g/L (▴), 18 g/L (⋄) and 20 g/L (□). High concentrations of aluminum, a common contaminant in biomining processes, inhibit the growth of sulfur oxidizing bacteria. We sought to evaluate if the kinetic advantages of using bioadaptable sulfur as a substrate counteract the effects of inhibition. Four flasks were prepared with bioadaptable sulfur, one for each aluminum concentration. Four more flasks were prepared with non-bioadaptable fine sulfur powder for each aluminum concentration. The inoculum was Licanantay strain DSM 17318, Acidithiobacillus thiooxidans strain property of BioSigma S.A., protected in the patent application CL 2101-2005 and in U.S. Pat. No. 7,700,343. The cellular concentration of the inoculum was estimated by optical counting at 5 10⁹ cells/mL.

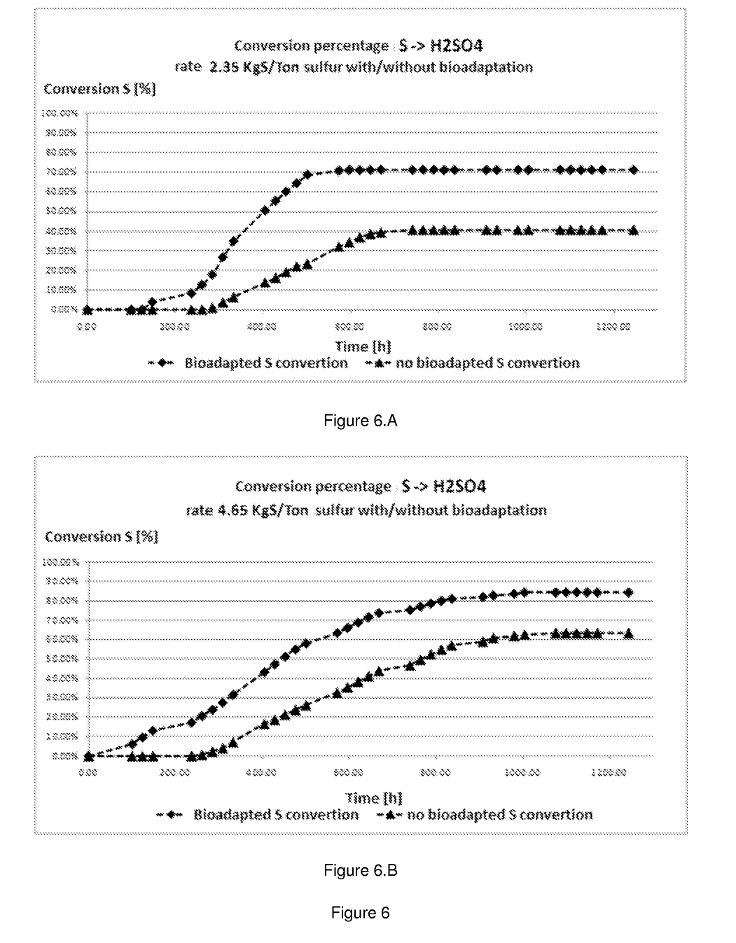

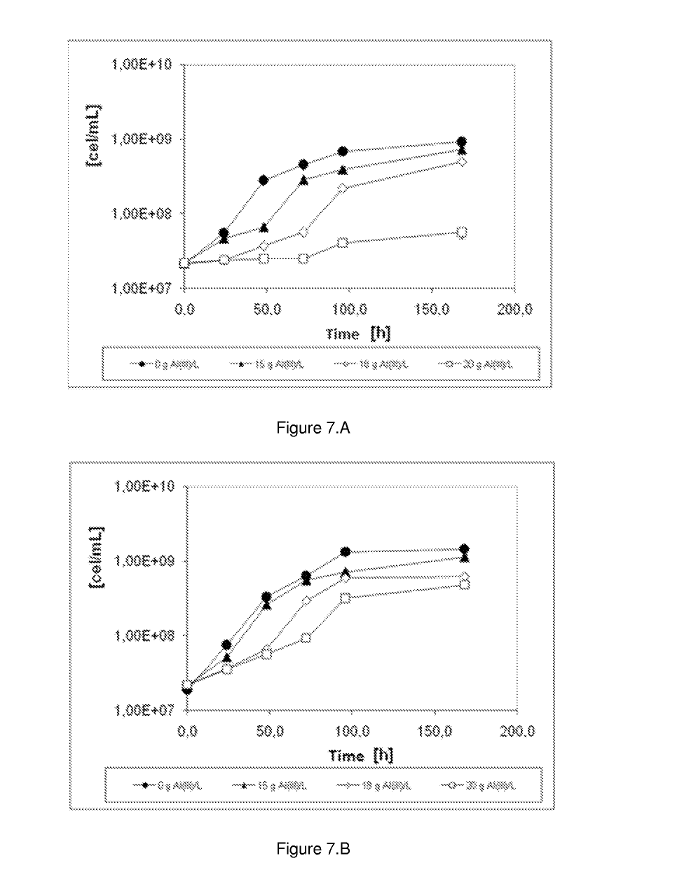

FIG. 6 shows the growth curves that were obtained by optical biomass counting. FIG. 6A shows the control, using non-bioadaptable fine sulfur powder, for the different concentrations of the toxic agent. As the aluminum concentration increases in solution, biomass growth is clearly hindered. There is virtually no increase in biomass for the highest aluminum concentration of 20 g/L (□): the biomass concentration is significantly less than the control () at 170 hours. FIG. 6.B shows the same reactions using bioadaptable sulfur as the substrate, obtained by the method and system of the invention. The inhibitory effect of aluminum sulfate is less relative to the inhibition when non-bioadaptable sulfur is used as substrate. The highest concentration of aluminum 20 g/L (□) with bioadaptable sulfur, shows an increase in biomass, which is slightly less than the control without aluminum () at 170 hours.

From the previous results, we can see advantages of using bioadaptable sulfur obtained by the method of the invention, instead of using fine sulfur powder for bioreactors or growth columns/cells on packed beds. The bioadaptable sulfur can be easily used as a homogenous aqueous dispersion, or as a slurry with easy homogenization on a solid bed. This makes the sulfur bioavailable more rapidly for sulfur oxidizing bacteria, which accelerates biomass growth in bioreactors and columns/cells.

1. A system for sulfur bioadaptation in a stable dispersion form for industrial bioreactor feeding, WHEREIN the following is included:
a) a tank with a liquid medium made up of a supernatant dilution or part of a sulfur oxidizing bacterial culture;
b) a recirculating pump fed from the tank, and
c) an eductor which is connected at its input to the recirculation pump, and connected at its output back to the tank, closing the recirculation circuit, and there is a supply of fine sulfur powder in the eductor's feeding path.

2. A system for sulfur bioadaptation according to claim 1, WHEREIN it contains a means for directing flow connected to the recirculation pump output, which allows to selectively direct flow.

3. A system for sulfur bioadaptation according to claim 2, WHEREIN the means for directing flow corresponds to a 3-way valve or a set of two valves each controlling flow entry to the corresponding circuit directions.

4. A method for sulfur bioadaptation in a stable dispersion form for industrial bioreactor feeding which includes these steps:
a) initiating recirculation within the tank which consists of a liquid medium containing a supernatant dilution or a sulfur oxidizing bacterial culture, through a recirculation pump, and
b) connecting a supply of fine sulfur powder to an eductor connected to the recirculation pump at its input, and connecting its input back into the tank.

5. A method for sulfur bioadaptation according to claim 4, WHEREIN the additional steps are included:
c) connecting a means for directing flow at the recirculation pump outlet which allows to selectively direct the flow, and
d) selectively switch the flow by the means for directing flow, to operate the eductor through the recirculation flow or to direct the flow directly from the recirculation pump to the tank.
